(19) United States
(12) Patent Application Publication
(10) Pub. No.: US 2026/0051400 A1
(43) Pub. Date: Feb. 19, 2026

(54) MEDICAL INFORMATION PROCESSING APPARATUS AND METHOD

(71) Applicant: CANON MEDICAL SYSTEMS CORPORATION, Otawara-shi (JP)

(72) Inventors: Hamish MACKINNON, Edinburgh (GB); Alison O'NEIL, Edinburgh (GB); Francesco Dalla SERRA, Edinburgh (GB); Antanas KASCENAS, Edinburgh (GB)

(73) Assignee: CANON MEDICAL SYSTEMS CORPORATION, Otawara-shi (JP)

(21) Appl. No.: 18/804,635

(22) Filed: Aug. 14, 2024

(51) Int. Cl. G16H 50/20; G16H 30/40

(52) U.S. Cl. CPC G16H 50/20; G16H 30/40

(57) ABSTRACT

A medical information processing apparatus comprising processing circuitry configured to: obtain initial medical data associated with ground truth medical text data, wherein the ground truth medical text data comprises at least partially unstructured medical text data, wherein the initial medical data comprises structured medical text data generated or otherwise obtained from the ground truth medical text data and/or medical image data associated with the ground truth medical text data; generate synthetic medical text data from the initial medical data using a synthetic text generator, wherein the generated synthetic medical text data comprises at least partially unstructured data; obtain predicted ground truth label information by performing a classification process on the ground truth medical text data; obtain further label information comprising at least one of annotated ground truth label information for the ground truth medical text data and/or synthetic label information for the generated synthetic medical text data; perform at least one further data processing task using the predicted ground truth label information and the further label information.

### FIELD

Embodiments described herein relate generally to a method and apparatus for processing medical information, for example, for generating synthetic medical text data. Certain embodiments relate to, for example, a method and apparatus for training and/or refining a text generation model and/or for evaluating the output of said models.

### BACKGROUND

It is known to use machine learning model to generate text. In particular, it is known to use machine learning to automatically generate radiology reports. With current text generator artificial intelligence/machine learning language models, the style and grammatical accuracy tends to be good but the factual accuracy may not be. As a result, generated text may contain clinical content errors in the form of, for example, hallucinations (additional untrue facts), omissions (missed facts) or wrong information (changed facts). Hallucinations, omissions and wrong information are clearly undesirable. These issues may be caused by using a standard pre-training objective which is next token prediction, with a cross-entropy loss function based on linguistic similarity to the ground truth text (i.e. a perfect score is achieved when the wording matches exactly) rather than semantic similarity (i.e. rewarding similar meaning even when different wording is used).

The following example is provided to demonstrate how a generated report may sound plausible but contain different types of clinical content errors. An example of a generated chest x-ray radiology report is taken from Dalla Serra, F., Clackett, W., Mackinnon, H., Wang, C., Deligianni, F., Dalton, J. and O'Neil, A. Q., 2022 November, “Multimodal Generation of Radiology Reports using Knowledge-Grounded Extraction of Entities and Relations”, in AACL-IJCNLP proceedings. The original report reads “Large left lower lobe opacity is present. There does not appear to be significant mediastinal shift. There is no pneumothorax. The cardiac silhouette is not definitively identified and not fully evaluated. The mediastinal contours are unremarkable”. The generated report reads “PA and lateral views of the chest were reviewed and compared to the prior studies. A right pleural effusion has increased in size since the prior study. The left lung is clear. There is no pneumothorax.” On comparison between the original report and the generated report, there are clearly clinical errors in the form of the omission of left lower lobe opacity, and the hallucination of pleural effusion.

Lovelace and Mortazavi (“Learning to generate clinically coherent chest X-ray reports”, in Findings of the Association for Computational Linguistics: EMNLP 2020 (pp. 1235-1243), 2020 November) have proposed to supervise training of a text generation model using a loss function modelling clinical accuracy. This is referred to as a clinical coherence loss. Clinical labels for the presence or absence of 14 pathologies are predicted for the generated text by a neural network classifier (“differentiable CheXpert”), and prediction errors are then backpropagated through to the generative model. A differentiable neural network is used as the classifier so that prediction errors can be backpropagated through this model and hence through the target generative model during training.

CheXpert is a large public dataset for chest radiograph interpretation, consisting of 224,316 chest radiographs of 65,240 patients together with associated radiology reports. Each radiology report is labelled using an automated labeller to extract observations from the free text radiology reports to be used as structured labels. The labels correspond to the status of each of the 14 types of observation i.e. positive, negative, uncertain, and not mentioned. The labels can be used as ground truth labels for the radiograph images, together forming training data for training of a supervised or at least semi-supervised model.

### BRIEF DESCRIPTION

Embodiments are now described, by way of non-limiting example, and are illustrated in the following figures, in which:

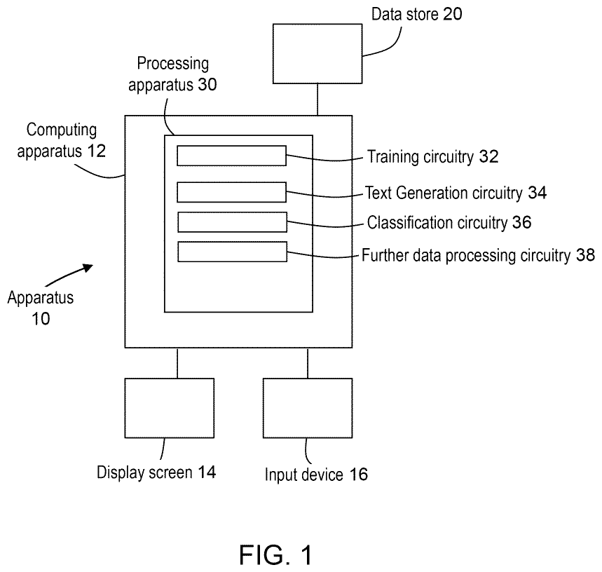

FIG. 1 is a schematic diagram of an apparatus according to an embodiment;

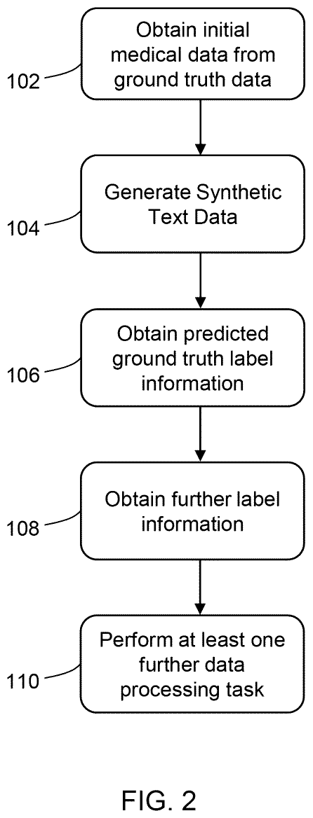

FIG. 2 is a flow chart illustrating in overview a method in accordance with an embodiment;

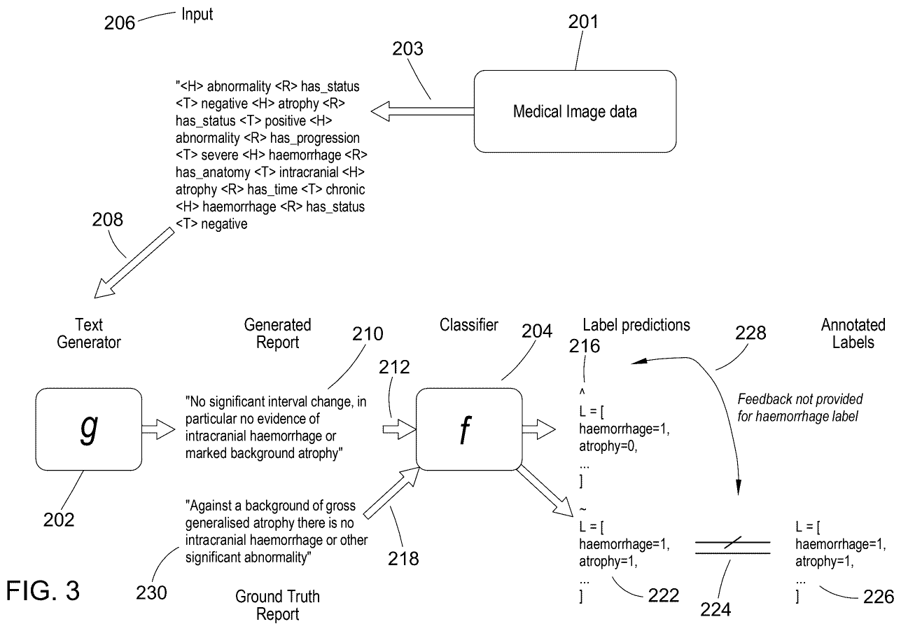

FIG. 3 is a flow chart illustrating in overview a method in accordance with an embodiment;

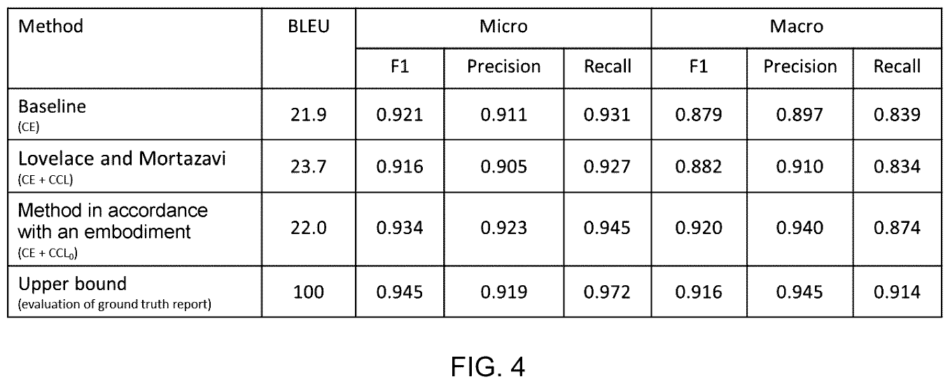

FIG. 4 illustrates results obtained using the method of FIG. 3.

### DETAILED DESCRIPTION

Certain embodiments provide an apparatus, for example, a medical information processing apparatus, comprising processing circuitry configured to: obtain initial medical data associated with ground truth medical text data, wherein the ground truth medical text data comprises at least partially unstructured medical text data, wherein the initial medical data comprises structured medical text data generated or otherwise obtained from the ground truth medical text data and/or medical image data associated with the ground truth medical text data; generate synthetic medical text data from the initial medical data using a synthetic text generator, wherein the generated synthetic medical text data comprises at least partially unstructured data; obtain predicted ground truth label information by performing a classification process on the ground truth medical text data, wherein the classification process uses a differentiable model; obtain further label information comprising at least one of annotated ground truth label information for the ground truth medical text data and/or synthetic label information for the generated synthetic medical text data; perform at least one further data processing task using the predicted ground truth label information and the further label information.

Certain embodiments relate to a method comprising obtaining initial medical data associated with ground truth medical text data, wherein the ground truth medical text data comprises at least partially unstructured medical text data, wherein the initial medical data comprises structured medical text data generated or otherwise obtained from the ground truth medical text data and/or medical image data associated with the ground truth medical text data; generating synthetic medical text data from the initial medical data using a synthetic text generator, wherein the generated synthetic medical text data comprises at least partially unstructured data; obtaining predicted ground truth label information by performing a classification process on the ground truth medical text data, wherein the classification process uses a differentiable model; obtaining further label information comprising at least one of annotated ground truth label information for the ground truth medical text data and/or synthetic label information for the generated synthetic medical text data; performing at least one further data processing task using the predicted ground truth label information and the further label information.

Certain embodiments relate to a non-transitory memory storing computer-readable instructions that are executable by a processor to: obtain initial medical data associated with ground truth medical text data, wherein the ground truth medical text data comprises at least partially unstructured medical text data, wherein the initial medical data comprises structured medical text data generated or otherwise obtained from the ground truth medical text data and/or medical image data associated with the ground truth medical text data; generate synthetic medical text data from the initial medical data using a synthetic text generator, wherein the generated synthetic medical text data comprises at least partially unstructured data; obtain predicted ground truth label information by performing a classification process on the ground truth medical text data, wherein the classification process uses a differentiable model; obtain further label information comprising at least one of annotated ground truth label information for the ground truth medical text data and/or synthetic label information for the generated synthetic medical text data; perform at least one further data processing task using the predicted ground truth label information and the further label information.

An apparatus 10 according to an embodiment is illustrated schematically in FIG. 1. The apparatus 10 may be referred to as a medical information processing apparatus. Medical information may be provided, for example, in the form of medical data. The apparatus 10 comprises a computing apparatus 12, which in this case is a personal computer (PC) or workstation. The computing apparatus 12 is connected to a display screen 14 or other display device, and an input device or devices 16, such as a computer keyboard and mouse.

The computing apparatus 12 receives initial medical data from a data store 20. In alternative embodiments, computing apparatus 12 may receive the medical data from one or more further data stores (not shown) instead of or in addition to data store 20. For example, the computing apparatus 12 may receive medical data from one or more remote data stores (not shown) which may form part of a Picture Archiving and Communication System (PACS) or other information system.

Computing apparatus 12 provides a processing resource for automatically or semi-automatically processing medical text data. Computing apparatus 12 comprises a processing apparatus 30. The processing apparatus 30 comprises training circuitry 32 for training a text generation model and/or a classification model; text generation circuitry 34 configured to generate text data using the trained text generation model; classification circuitry 36 configured to perform a classification process using the trained classification model.

The processing apparatus 30 also includes further data processing circuitry 38 configured to perform at least one further data processing task. In some embodiments, the data processing circuitry is configured to perform fine tuning of the text generation model and/or classification model. Fine tuning of the models may also be referred to as further training or refining of the models. In some embodiments, the at least one further data processing task includes an evaluation of the output of the text generation model and/or the classification model. In some embodiments, the at least one further data processing task includes calculating a loss function, for example, as part of a training and/or refining process. In some embodiments, the training circuitry comprises at least a first training circuitry for training the text generation model and a second training circuitry for training the classification model.

In the present embodiment, the circuitries 32, 34, 36, 38 are each implemented in processing apparatus 30 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. For clarity, such components are not shown in FIG. 1.

The apparatus of FIG. 1 is configured to perform a method of an embodiment as shown in FIG. 2.

Prior to method step 102, a synthetic text generator is obtained. The synthetic text generator corresponds to a procedure based on a text generation model. Obtaining the synthetic text generator therefore included performing a training process in which the text generation model is trained using training circuitry 32. The training of the synthetic text generation model may include a general training phase, in which the text generation model is trained without specific reference to medical text data. The synthetic text generation model may be a large language model and/or other deep learning derived language model. A deep learning model may be understood as a model based on artificial neural networks having multiple layers. In some embodiments, a pre-trained or at least partially trained synthetic text generator model is obtained. In some embodiments, the synthetic text generator is not trained.

As a non-limiting example, to train the text generation model, a large volume of training data is obtained. It will be understood that the training data may include clinical text training data and also text data that is not considered medical text data. The text data are used to train an embedding using a standard pre-training task, for example word2vec. The standard pre-training task comprises training an embedding using a large corpus of text. In a representation that is based on embeddings, the text is considered to be a set of word tokens. A word token may be, for example, a single word, a part of a word, or a group of words. A respective embedding vector is assigned to each word token. The standard pre-training task trains the embedding. Multiple iterations of the standard pre-training task may be performed, with the embedding updated at each iteration. Embedding vectors capture semantic similarity between word tokens in a multi-dimensional embedding space. An embedding may be a dense (vector) representation of a semantic space of words. An output of the training process is a trained embedding which comprises a respective vector representation of each of a plurality of words from the training corpus.

During initial training, the synthetic text generator may be initially trained on a large volume of data that includes non-medical data. The generated synthetic text may be stylistically and grammatically correct, however, the output may contain clinical errors including, but not limited to, additional fabricated information (referred to as hallucinations), omissions, or incorrect information. As a result, while the generated output text will likely be stylistically and grammatically correct (such that the output scores highly in such tests) it is clearly undesirable to output text that contains clinical errors.

In some embodiments, the synthetic text generation model may be initially trained using a first training objective and, as described in the following, further trained or refined using a second training objective. For example, the first training objective may be based on achieving a level of grammatical similarity between the input and output and the training may use a suitable loss function (for example, a cross-entropy based loss function). As described in the following, the further training and refinement of the model is based on a second training objective.

The text generation model may be, for example, a transformer model. However, any suitable text generation model configured to generate free text, or at least partially unstructured text, may be used. The text generator may be a BART model. BART models are described, for example, in Lewis et al. “BART: Denoising Sequence-to-Sequence Pre-training for Natural Language Generation, Translation, and Comprehension”, ACL 2020.

In addition, a classifier model is trained, for example, using the training circuitry 32. The classifier may comprise any differentiable classifier. As a non-limiting example, the training of the classifier involves receiving an annotated clinical text corpus that comprises, for example, a plurality of annotated radiology reports. Annotations of the radiology reports may comprise, for example, classification labels. The annotated clinical text corpus is used as training input for training a deep learning model, in this case a classifier. The deep learning model is trained to provide an output for each of a plurality of classes. The outputs may include, for each class, a score indicating a probability or likelihood for the class. The score may be a document or sentence-level score. The model outputs may include, for each class, a word-level attention weighting.

In the present embodiment, a differentiable classifier is described. In further embodiments, a non-differentiable classifier may be used subject to modifications of the loss function.

In one example, the classifier is trained to obtain predictions for a certain set of labels for each sentence of the radiology report and/or for the radiology report as a whole. Each label relates to a corresponding medical term, for example, a finding or impression. For example, labels may include haemorrhage and tumour. The classifier may be trained to classify each sentence or report to say whether each of haemorrhage and tumour is present.

For each sentence, each of the labels is classified in one of a plurality of certainty classes. The certainty classes include positive, uncertain and negative. A classification in the positive certainty class is made when the model determines from the sentence that the finding or impression that is represented by the label is present in the image. A classification in the negative certainty class is made when the model determines from the sentence that the finding or impression that is represented by the label is not present in the image. A classification in the uncertain certainty class is made when the model determines from the sentence that there is uncertainty over the presence of the finding or impression in the image. For example, the sentence may suggest that the finding or impression may be present without providing a strong enough indication to be classified as positive. In the following embodiments, the labels are from a set of 14 types of observation.

In some embodiments, the classifier is substantially as described in Schrempf et al. “Templated text synthesis for expert-guided multi-label extraction from radiology reports” MAKE 2021, 3(2), the contents of which are hereby incorporated by reference.

Returning to the method of FIG. 2, at stage 102, initial medical data is obtained. The initial medical data is obtained from initial medical image data that is provided to together with at least partially unstructured medical text data. In the following embodiments, the initial medical data are obtained from initial medical images in the form of one or more radiology images. The radiology images are provided with initial ground truth data, in the form of a human generated radiology report, referred to as a ground truth radiology report. The ground truth radiology report is provided together with annotated labels that are referred to as ground truth label information. The ground truth radiology report will be understood as having portions of unstructured medical text data.

As described with reference to FIG. 3, in some embodiments, the initial medical data is structured data obtained from the initial medical images. The initial medical text data comprises a sequence of unordered triplets as input. These are provided in the format Entity->Relationship->Attribute, expressed in text as “<H> entity <R> relationship <T> attribute”). Further detail on generation of the structured data is provided with reference to FIG. 3.

Other suitable initial medical data may be used. In the present embodiment, the initial medical data may comprise medical image data and the associated text data are, for example, corresponding radiology reports for the images. In alternative embodiments, the structured medical text data is obtained from unstructured medical text data. The initial medical data is therefore associated with ground truth data.

At stage 104, synthetic medical text data is generated using the synthetic text generator. The synthetic text generator is configured to receive the initial medical text data, for example, the structured initial medical data described above, and to generate a free form synthetic medical text in the form of a synthetic radiology report. The generated synthetic text will be partially unstructured in that it contains one or more unstructured portions. In other embodiments, the synthetic text generator is configured to receive medical image data and generate at least partially unstructured synthetic text.

At stage 106, predicted ground truth label information is obtained by performing a classification process on the initial ground truth data. In this embodiment, the classifier is applied to ground truth radiology reports to obtain the predicted ground truth label information.

At stage 108, further label information is obtained. The further label information includes synthetic text label information obtained by performing a further classification process on the generated synthetic text, from step 104. In addition or alternatively, the further label information includes the stored ground truth label information that was initially provided together with the initial medical data. As described above, the ground truth label information includes annotations for the initial radiology reports. In some embodiments, the further label information includes both the stored ground truth label information and the synthetic label information.

At stage 110, a further data processing task is performed using at least the obtained predicted ground truth label information and the further label information. In some embodiments, the further data processing task a model training, refining or fine tuning task. For example, the further data processing task may be a model fine tuning process in which the text generator is fine-tuned based on at least the predicted ground truth and the further label information. In some embodiments, the further data processing task may include an error back propagation process in which clinical errors are identified and fed back to the classifier and/or text generation model.

In some embodiments, the further data processing task may be an evaluation task in which the generated synthetic text is evaluated by comparing the predicted ground truth labels with the initial ground truth labels to obtain a score or other evaluation measure. Such an evaluation measure may indicate the clinical accuracy of the generated text.

FIG. 3 depicts a flowchart showing in overview a method of training and/or fine-tuning a synthetic text generator, in accordance with an embodiment. FIG. 3 depicts a text generator 202 and a classifier 204 used in the method. The text generator substantially corresponds to the text generator described with reference to FIG. 2. The classifier corresponds to the classifier described with reference to FIG. 2.

As described with reference to FIG. 2, at an initial stage, a trained or at least partially trained text generator 202 is obtained.

Initial medical data 206 are obtained. In this embodiment, the initial medical text data are structured medical data. In the present embodiment, the initial structured medical data are obtained from a radiological image or other medical image data 201. The initial structured medical image data is obtained using a triplets extractor, at step 203 to obtain the initial medical text data. The medical image data 201 corresponds to initial ground truth data, in the form of a ground truth report 220. In embodiments, the ground truth report 220 and corresponding medical image data 201 are obtained from a database.

The initial medical text data comprises a sequence of triplets as input, where the triplets may be ordered or unordered. These are provided in the format Entity->Relationship->Attribute, expressed in text as “<H> entity <R> relationship <T> attribute”). The structured data is obtained from the initial image using a known image classification model. An example, of a known classification model is described at FIG. 1) of Dalla Serra, F., Clackett, W., Mackinnon, H., Wang, C., Deligianni, F., Dalton, J. and O'Neil, A. Q., 2022 November, “Multimodal Generation of Radiology Reports using Knowledge-Grounded Extraction of Entities and Relations”, in AACL-IJCNLP proceedings.

In this example, the initial medical data reads: “<H> abnormality <R> has_status <T> negative <H> atrophy <R> has_status <T> positive <H> abnormality <R> has_progression <T> severe <H> haemorrhage <R> has_anatomy <T> intracranial <H> atrophy <R> has_time <T> chronic <H> haemorrhage <R> has_status <T> negative”

In the present embodiment, the ground truth radiology report 220 reads: “Against a background of gross generalised atrophy there is no intracranial haemorrhage or other significant abnormality”.

At stage 208, the structured initial medical data is provided to the synthetic text generator 202. The synthetic text generator 202 processes the initial medical data and generates, as an output, a generated medical report 210. The generated medical report 210 includes partially unstructured text data. The medical report 210 is an example of synthetic text data generated by the synthetic text generator 202. In this example, the generated synthetic text has a portion that includes the unstructured text reading: “No significant interval change, in particular no evidence of intracranial haemorrhage or marked background atrophy”. Obtaining a generated report from an initial medical image data, as described above, may be referred to as a two-step pipeline.

At stage 212, a first classification process is performed using the differentiable classifier 204. The first classification process comprises applying classifier 204 to the synthetic text 210 to produce first label information 216. The label information obtained by applying the classifier to the synthetic text is referred to as synthetic label information and is to be distinguished from the label information obtained at stage 218 and the initial ground truth label information 226. In the present embodiment, the synthetic label information reads \hat{L}=[haemorrhage=1, atrophy=0, . . . ].

At stage 218, a second classification process is performed. The second classification process comprises applying classifier 204 to initial ground truth data in the form of the ground truth report 220 to produce second label information 222. The second label information 222 is referred to as predicted ground truth label information and is to be distinguished with the ground truth label information that is initially available together with the ground truth radiology report i.e. annotation labels obtained following a human annotation process of the radiology report. In the example of the Figure, the predicted ground truth label information reads \tilde{L}=[haemorrhage=1, atrophy=1, . . . ].

In the present embodiment, the first classification process produces first label information 216 comprising synthetic label information for the generated synthetic medical text 210 and the second classification process produces second label information 222 in the form of ground truth label information 222.

At stage 224, a comparison process is performed using predicted ground truth label information 222. In the present embodiment, the comparison process provides clinical content error information using the predicted ground truth label information 222. In accordance with embodiments, the ground truth label information may be used to obtain clinical error information using a number of different methods. In the present embodiment, the clinical error information is obtained by comparing the predicted ground truth label information 222, as obtained by applying the classifier to the initial ground truth report 220, with the initial ground truth label information 226 i.e. the human annotated labels. At this stage, the classification of the ground truth report 220 is compared to the human annotated labels.

In the present embodiment, the ground truth label information reads L=[haemorrhage=0, atrophy=1, . . . ]. In this example, the label information for the label haemorrhage does not agree based on a comparison between the ground truth label information and the predicted ground truth label information. As such it is clear that the classifier, which predicted the presence of a haemorrhage given the ground truth report as input, has failed to extract the correct facts. As the classifier does not seem to be reliable errors based on the classifier predictions should not be fed back to the generator model based on the classifier's prediction.

In some embodiments, clinical content error information for generated synthetic text may be identified in the generated synthetic text by performing a comparison between the predicted ground truth labels and the true ground truth labels. In some embodiments, a predicted label that does not match an annotated labels is indicative of a clinical content error. A mismatched label may represent a difference in clinical content of the generated text. A mismatched label may represent a hallucination, omission or incorrect clinical information. A mismatched label may represent clinical inaccuracy in the generated synthetic text.

At stage 228, one or more further data processing tasks are performed. In the present embodiment, a model fine tuning task is performed, in which one or more weights or other model parameters of the text generation model and/or classifier are modified based on the obtained predicted ground truth label information, and the ground truth label information and the synthetic label information. In the example of FIG. 3, the error information for the haemorrhage label indicates that feedback for this label should be discarded and not provided. Further detail regarding the fine tuning of the text generation model is provided with reference to the following embodiment.

In further detail, step 228 includes an error propagation process, in which the loss function for the text generator uses the identified clinical errors to modify and/or refine one or more weights of the text generation model. In the described embodiments, the backpropagation process may refer to calculating one or more suitable terms in the loss function.

As described in the following, the fine-tuning of the text generation model included calculating a loss function which penalised likely errors in the generated text. For example, as described in the following, when predicted ground truth labels do not match the initial ground truth labels from the annotated ground truth, the contribution to the loss function for that label in the fine-tuning of the text generation model is discarded and/or suppressed.

As a further example, the clinical coherence loss function may be extended and/or modified to provide feedback from soft labels. Soft label output from the classifier is distinguished from hard labels in that the soft labels output includes scores representing probabilities that a label is corrected, in contrast to the hard label information that represents a predicted label (for example, following a thresholding process). As an example, rather than applying the consistency loss function on hard predicted ground truth labels and the initial, annotated ground truth labels, as described above, the loss function is applied to the output logits of the classifier. In such an embodiment, a loss function having a contribution based on that soft error information is calculated.

As described in the following, the fine-tuning of the text generation model included calculating a loss function which penalised likely errors in the generated text. For example, as described in the following, when predicted ground truth labels do not match the initial ground truth labels from the annotated ground truth, the contribution to the loss function for that label in the fine-tuning of the text generation model is discarded and/or suppressed.

A further embodiment is described in detail in the following. The following embodiment describes a clinical coherence loss function that uses synthetic label information and ground truth label information. In embodiments, label information includes one or more labels. Each label corresponds to data representing a number of classes. In some embodiments, the label information also includes corresponding scores representing a probability, or likelihood, that the data is represented by the label. In some embodiments, the scores represent the probability or likelihood that the data belongs to the class of that label. In some embodiments, the label information includes logits corresponding to each label.

In the described embodiments, a comparison between label information is described. In some embodiments, the comparison includes determining common labels, one or more labels that do not match and/or labels present in one set of labels and not present in another set of labels. Labels that do not match or are missing, may be indicative of clinical errors, such as omissions, hallucinations or factually incorrect information. In some embodiments, the label information may also include score associated with it for example, representing probabilities or likelihood. This label information may also be referred to as soft label information. Comparison of soft label information may include calculating one or more comparison functions using the scores, such as a cross-entropy function.

The text generator g (corresponding to text generator 202) can be considered as a function mapping data to output logits. The text labeller f (corresponding to classifier 204) can be considered as a function mapping token embedding to output labels. The output logits can be considered as an output or intermediate value of the text generator model. The logits can be considered as unscaled output of an intermediate layer of the model.

$$g : Input Text / Image / Data \to Output Logits .$$

$$f : Token Embeddings \to Output Labels .$$

During training of the text generator, given the input Text/Image/Data (x) the result of the text generator g consists of the Output Logits:

$$π = g ( x ) ∈ {ℝ}^{K \times m}$$

with K being the vocabulary length and m the length of the target output sequence.

The Output Logits x are then input to a GumbelSoftmax function to obtain a set of predicted tokens ŷ.

$$\hat{y} = GumbelSoftmax ( π ) ∈ {ℝ}^{K \times m}$$

The GumbelSoftmax function is a differentiable function that returns the Predicted Tokens ŷ, expressed as m one-hot vectors with the index of the most likely predicted token marked as 1 and 0s otherwise. These are multiplied with the word embedding matrix W∈ [symbol] ^{K×d} (with d being the dimension of each embedded token) to obtain the Predicted Token Embeddings:

$$\hat{w} = {W}^{T} \hat{y} ∈ {ℝ}^{d \times m}$$

an encoded representation of the generated text.

Finally, the text labeller f takes the Predicted Token Embeddings ŵ, as input and returns the predicted probabilities \hat{p}(l) associated with the generated text:

$$\hat{p} = f ( \hat{w} )$$

In this example, the consistency loss function & corresponds to a Cross Entropy loss function (CE) between the Predicted Labels T and the Ground Truth Labels l:

$$ℒ = CE ( \hat{p} ( l ) , l ) .$$

The loss function therefore represents a measure of similarity or match between ground truth label information and the predicted (synthetic) label information. The cross entropy loss function increases when ground truth labels agree with the synthetic labels and are lower when ground truth labels do not agree with the synthetic labels. As such the cross-entropy loss function allows a comparison between the synthetic labels and the ground truth labels. The loss functions allow clinical content information to be used to train and/or refine the text generation model.

In addition to the above method, the present embodiments include a modified loss function term. In some embodiments, the modified loss function corresponds to the above-described loss function (a cross-entropy function using the predicted synthetic label information and the ground truth label) but subject to constraints using predicted ground truth label information. As a first example, as described with reference to FIGS. 2 and 3, the classifier is applied to extract ground truth prediction label information. Using a ground truth report in tokenized form (represented as \tilde{y}) the predicted ground truth labels can be obtained:

$$\tilde{w} = {W}^{T} \tilde{y}$$

$$\tilde{p} ( l ) = f ( \tilde{w} )$$

The predicted ground truth labels are obtained by threshold predicted label probabilities with a threshold value (t). Typically a value of 0.5 is used.

$$\tilde{l} = [ \tilde{p} ( l ) > t ]$$

Using the predicted ground truth label information, the clinical coherence loss function described above can be modified or extended as follows.

As a first example, likely errors in the generated text can be discarded. For example, when the predicted ground truth labels \tilde{l} disagree with the initial ground truth labels l (also referred to as the annotated ground truth labels) any contribution to the consistency loss function for that label may be discarded. The loss function may be represented as:

$${ℒ}_{f} = CE ( \hat{p} ( l ) , l ) .$$

and subject to a constraint based on the predicted ground truth labels.

The piecewise function [symbol] _{f} has a non-zero contribution only when the value of a predicted ground truth label matches the value of the ground truth label.

$${ℒ}_{f} = \{ \begin{matrix}CE ( \hat{p} ( l ) , l ) & if l = \tilde{l} \\ 0 & otherwise\end{matrix}$$

As an alternative, the clinical coherence loss function may be extended and/or modified to provide feedback from soft labels. As an example, rather than applying the consistency loss function on predicted ground truth labels and the initial, annotated ground truth labels, as described above, the loss function is applied to the output logits of the text labeller f. In such an embodiment, the predicted logits \hat{π} are obtained from the tokenized synthetic text report ŷ (i.e. the predicted tokens) as:

$$\hat{π} = {f}^{- 1} ( \hat{y} )$$

Where f⁻¹ is the text labeller network without the final classification layer. Likewise, the predicted ground truth logits \tilde{π} are obtained from the tokenized ground truth report \tilde{y}:

$$\tilde{π} = {f}^{- 1} ( \tilde{y} ) .$$

The consistency loss is then computed as follows:

$${L}_{f} = \{ \begin{matrix}CE ( \tilde{π} , \hat{π} ) & if l = \tilde{l} \\ 0 & otherwise\end{matrix}$$

In this example, the consistency loss is combined with error discarding, as described above.

In the above example, predicted ground truth label information in the form of predicted labels \tilde{l} and associated scores in the above example, logits \hat{π} are used as an input to the loss function for training/refining and/or evaluation. It has been found that some errors may be false positives in that the classifier incorrectly extracts or does not extract a label from generated text. In addition, there may be disadvantages with using hard feedback from a correct label class in the form of a one-hot label vector. Such feedback provides limited information about relationships with other classes.

The coherence loss function can be modified/extended as set out above. As a first example, likely errors can be discarded. As a second example, a matching tool may give more granular feedback, for example by associating at a report level or sentence level, labels with sentences and words that should contain the label information. This provides heterogeneous feedback across the report. As a further example, a more complete source of clinical facts may be employed than the dedicated labeller is trained for, for example, by using a general entity/attribute extraction model (for example, a classifier trained to extract UMLS concepts). As a further example, the classifier's predictions for the ground truth report may be treated as true labels for a generated report.

In the above described embodiments, the cost function is zero or has reduced contribution when the initial ground truth information does not sufficiently match the predicted ground truth label information. The matching may be based on hard labels or soft labels. In some embodiments, a matching process is performed to determine which labels do not match. In some embodiments, for soft label information, a matching process is performed that determines a measure of distance between the different sets of label information.

FIG. 4 presents a table of results from the method described above. The reports are generated sentence-wise and evaluation of the reports performed on 463 sentences. The table shows results for the original training task with a cross-entropy loss for next token prediction (CE) in the first row. In the second row, the table show results for addition of a clinical coherence loss (CCL). In the third row, the table also shows results for a method in accordance with embodiments. The loss function of the method includes a modified clinical coherence loss CCL₀ in which the examples where the prediction on ground truth is discarded or zeroed out. The fourth row displays values for an upper bound.

A BLEU score is reported (Papineni et al., 2002). This metric represents a similarity in language between the generated and ground truth reports. Micro and Macro averages of precision, recall and F1 score are included in the table. As can be observed in the table, the proposed loss of CE+CCL₀ offers improvement in these metrics.

The above metrics are obtained by comparing detected labels to ground truth labels. For example, if all the ground truth labels are correctly predicted, this would yield a recall of 1. If some extra wrong instances (false positives) are predicted, that would lead to a poorer precision, lower than 1.

In the above described embodiments, the prediction of predicted ground truth label information allows likely errors on the generated text to be discarded. For example, if the classifier makes an error on the original text, we consider that it is more likely to make an error on the generated text, and can ignore feedback from those labels. In addition, soft label information can be used to feed back more informative error information. This may be based on distillation techniques.

In addition, or alternatively, to fine tuning the text generation model, further data processing tasks may be performed based on the predicted ground truth label information. As a first non-limiting example, the predicted ground truth label information may be used as part of a matching tool to give more granular feedback by associating labels of the synthetic generated text (report-level, sentence-level) with the sentences/words that should contain the label information i.e. heterogeneous feedback across the report. In such embodiments, the same loss function may be used together with stronger supervision. In such embodiments, the error signal is associated more closely with a portion of the text that gave rise to the error.

As a second non-limiting example, the predicted ground truth label information may supplement the source of clinical information than the classifier is trained for. For example, by using a general entity/attribute extraction model (for example, a classifier trained to extract UMLS concepts). In some embodiments, a general classifier (for example, a dual encoder (such as that described in “Fast and Effective Biomedical Entity Linking Using a Dual Encoder by Bhowmik et al.) may be used as the conduit for feeding back error signals from a wider range of concepts than used during training.

In a further example, the clinical content information are identified in the synthetic text and the identified portions of the text that include errors may be displayed to a user. In some embodiments, the output a matching process may be performed identified portions may be discarded from the generated synthetic text. In some embodiments, the identified portions are penalized.

In some embodiments, the classification may be performed on only a portion of the generated text to obtain label information for that portion of the text. This allows label information to be generated for more than one portion of the text. This may be at a sentence-level, for example. The method may include matching portions of the text based on their predicted labels and the labels of the ground truth text.

In addition, the predicted ground truth label information can be stored as ground truth data for the generated report. As such, the generated report and the predicted ground truth labels can be stored and then used as training data for a further unsupervised or at least semi-supervised learning procedure for training a further model. In such a training process, the predicted ground truth labels are treated as the correct labels for the generated report.

Certain embodiments are provided to perform a computer-implemented method comprising: a deep learning model for generating output; a differentiable labelling tool for extracting structured data from unstructured output; a dataset of (un) structured inputs and (un) structured ground truth outputs and a set of labels describing the factual content of the target output; in which during training, the differentiable labelling tool is applied to both the generated output and the ground truth outputs to generate label probabilities from which clinical content errors may be computed and fed back into the generator model via backpropagation.

The clinical content error may be fed back only for labels where the label predictions on the ground truth report match a set of ground truth labels annotated by human experts.

Soft clinical content errors may be fed back based on the difference between the generated and ground truth label probabilities. The soft clinical content errors may be fed back not using hard ground truth labels.

Unsupervised training may be performed based on using automatically generated ground truth labels assigned by a labelling tool.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

Certain embodiments relate to a medical information processing apparatus comprising processing circuitry configured to: obtain initial medical data associated with ground truth medical text data, wherein the ground truth medical text data comprises at least partially unstructured medical text data, wherein the initial medical data comprises structured medical text data generated or otherwise obtained from the ground truth medical text data and/or medical image data associated with the ground truth medical text data; generate synthetic medical text data from the initial medical data using a synthetic text generator, wherein the generated synthetic medical text data comprises at least partially unstructured data; obtain predicted ground truth label information by performing a classification process on the ground truth medical text data; obtain further label information comprising at least one of annotated ground truth label information for the ground truth medical text data and/or synthetic label information for the generated synthetic medical text data; perform at least one further data processing task using the predicted ground truth label information and the further label information.

The classification process may use a differentiable model.

The at least one further data processing task may comprise at least one of a), b), c):
a) refining and/or training the synthetic text generation model and/or classification process;
b) evaluating output of the synthetic text generator based on at least the predicted ground truth label information;
c) identifying clinical content error information for at least part of the generated synthetic text;
d) calculating a loss function dependent on at least the predicted ground truth label information.

The processing circuitry may be configured to determine a loss function, for example, a cross entropy or other function using at least the predicted ground truth label information and the further label information.

The loss function may have a zero value and/or has an at least reduced contribution when at least part of the initial ground truth label information does not sufficiently match at least part of the predicted ground truth label information.

The classification process may comprise applying a classifier, optionally a differentiable classifier, to extract structured data comprising label information from at least partially unstructured text data.

The further label information may comprise ground truth label information for the ground truth data and the processing resource is configured to perform a comparison process between the initial ground truth label information and the predicted ground truth label information, wherein the at least one further data processing task is based on at least said comparison process.

The predicted ground truth label information may comprise label information generated by applying the classifier to the ground truth text data and wherein the ground truth label information comprises human annotated, or otherwise annotated, labels

Performing the classification process on the ground truth medical text data may use a pre-trained classifier, for example, a differentiable classifier, and wherein the processing apparatus is configured to perform a classification process on the synthetic label information using the same classifier to obtain the synthetic label information.

The processing circuitry may be configured to obtain clinical content error information in the generated synthetic text using at least the initial ground truth label information, wherein the clinical content error information comprises or represents one or more of: inaccurate information, incorrect information or omitted information.

The ground truth medical text data may comprise real data, for example, a medical report and/or a clinical text corpus comprising a plurality of medical reports.

The label information may comprise classification labels, optionally wherein the label information comprises scores associated with said classification labels, wherein the scores comprise at least one of a logit, probability and/or likelihood.

The at least one further data processing task may comprise performing an error backpropagation process to feedback obtained clinical content error information to the synthetic text generator based on at least the predicted ground truth label information.

The error backpropagation process may be performed based at least on a label classification score representing a probability and/or likelihood of the label.

The synthetic text generation model may comprise a deep learning model, for example, a transformer model or neural network model.

The processing circuitry may be configured to store the predicted ground truth label information together with the generated synthetic report as ground truth label information for the generated synthetic report. The processing circuitry may be configured to use the predicted ground truth label information and generated synthetic report as training data for a further at least partially unsupervised training process for a further model.

The at least one further data processing task may comprise identifying one or more portions of the synthetic generated text that include errors and discarding and/or otherwise penalizing said one or more identified portions of the synthetic generated text

The processing circuitry may be further configured to match and/or determine one or more relationships between the predicted ground truth label information and parts of the synthetic generated text data.

Certain embodiments relate to a method comprising: obtaining initial medical data associated with at ground truth medical text data, wherein the ground truth medical text data comprises at least partially unstructured medical text data, wherein the initial medical data comprises structured medical text data generated or otherwise obtained from the ground truth medical text data and/or medical image data associated with the ground truth medical text data; generating synthetic medical text data from the initial medical data using a synthetic text generator, wherein the generated synthetic medical text data comprises at least partially unstructured data; obtaining predicted ground truth label information by performing a classification process on the ground truth medical text data, wherein the classification process uses a differentiable model; obtaining further label information comprising at least one of annotated ground truth label information for the ground truth medical text data and/or synthetic label information for the generated synthetic medical text data; performing at least one further data processing task using the predicted ground truth label information and the further label information.

Certain embodiments relate to a non-transitory memory storing computer-readable instructions that are executable by a processor to: obtain initial medical data associated with ground truth medical text data, wherein the ground truth medical text data comprises at least partially unstructured medical text data, wherein the initial medical data comprises structured medical text data generated or otherwise obtained from the ground truth medical text data and/or medical image data associated with the ground truth medical text data; generate synthetic medical text data from the initial medical data using a synthetic text generator, wherein the generated synthetic medical text data comprises at least partially unstructured data; obtain predicted ground truth label information by performing a classification process on the ground truth medical text data, wherein the classification process uses a differentiable model; obtain further label information comprising at least one of annotated ground truth label information for the ground truth medical text data and/or synthetic label information for the generated synthetic medical text data; perform at least one further data processing task using the predicted ground truth label information and the further label information.

1. A medical information processing apparatus, comprising processing circuitry configured to:
obtain initial medical data associated with ground truth medical text data, wherein the ground truth medical text data comprises at least partially unstructured medical text data, wherein the initial medical data comprises structured medical text data generated or otherwise obtained from the ground truth medical text data and/or medical image data associated with the ground truth medical text data;
generate synthetic medical text data from the initial medical data using a synthetic text generator, wherein the generated synthetic medical text data comprises at least partially unstructured data;
obtain predicted ground truth label information by performing a classification process on the ground truth medical text data;
obtain further label information comprising at least one of annotated ground truth label information for the ground truth medical text data and/or synthetic label information for the generated synthetic medical text data; and
perform at least one further data processing task using the predicted ground truth label information and the further label information.

2. The apparatus of claim 1, wherein the classification process uses a differentiable model.

3. The apparatus of claim 1, wherein the at least one further data processing task comprises at least one of:
a) refining and/or training the synthetic text generation model and/or classification process;
b) evaluating output of the synthetic text generator based on at least the predicted ground truth label information;
c) identifying clinical content error information for at least part of the generated synthetic text; or
d) calculating a loss function dependent on at least the predicted ground truth label information.

4. The apparatus of claim 1, wherein the processing circuitry is configured to determine a loss function using at least the predicted ground truth label information and the further label information.

5. The apparatus of claim 4, wherein the loss function has a zero value and/or has an at least reduced contribution when at least part of the initial ground truth label information does not sufficiently match at least part of the predicted ground truth label information.

6. The apparatus of claim 1, wherein the classification process comprises applying a classifier to extract structured data comprising label information from at least partially unstructured text data.

7. The apparatus of claim 1, wherein the further label information comprises ground truth label information for the ground truth data and the processing resource is configured to perform a comparison process between the initial ground truth label information and the predicted ground truth label information, wherein the at least one further data processing task is based on at least said comparison process.

8. The apparatus of claim 7, wherein the predicted ground truth label information comprises label information generated by applying the classifier to the ground truth text data and wherein the ground truth label information comprises human annotated, or otherwise annotated, labels

9. The apparatus of claim 1 wherein performing the classification process on the ground truth medical text data uses a pre-trained classifier, and wherein the processing apparatus is configured to perform a classification process on the synthetic label information using the same classifier to obtain the synthetic label information.

10. The apparatus of claim 1, wherein the processing circuitry is configured to obtain clinical content error information in the generated synthetic text using at least the initial ground truth label information, wherein the clinical content error information comprises or represents one or more of: inaccurate information, incorrect information, or omitted information.

11. The apparatus of claim 1, wherein the ground truth medical text data comprises a medical report and/or a clinical text corpus comprising a plurality of medical reports.

12. The apparatus of claim 1, wherein the label information comprises classification labels, wherein the label information comprises scores associated with said classification labels, wherein the scores comprise at least one of a logit, probability, and/or likelihood.

13. The apparatus of claim 1, wherein the at least one further data processing task comprises performing an error backpropagation process to feedback obtained clinical content error information to the synthetic text generator based on at least the predicted ground truth label information.

14. The apparatus of claim 13, wherein the error backpropagation process is performed based at least on a label classification score representing a probability and/or likelihood of the label.

15. The apparatus of claim 1, wherein the synthetic text generation model comprises a deep learning model.

16. The apparatus of claim 1, wherein the processing circuitry is configured to store the predicted ground truth label information together with the generated synthetic report as ground truth label information for the generated synthetic report, using the predicted ground truth label information and generated synthetic report as training data for a further at least partially unsupervised training process for a further model.

17. The apparatus of claim 1, wherein the at least one further data processing task comprises identifying one or more portions of the synthetic generated text that include errors and discarding and/or otherwise penalizing said one or more identified portions of the synthetic generated text

18. The apparatus of claim 1, wherein the processing circuitry is further configured to match and/or determine one or more relationships between the predicted ground truth label information and parts of the synthetic generated text data.

19. A method, comprising:
obtaining initial medical data associated with at ground truth medical text data, wherein the ground truth medical text data comprises at least partially unstructured medical text data, wherein the initial medical data comprises structured medical text data generated or otherwise obtained from the ground truth medical text data and/or medical image data associated with the ground truth medical text data;
generating synthetic medical text data from the initial medical data using a synthetic text generator, wherein the generated synthetic medical text data comprises at least partially unstructured data;
obtaining predicted ground truth label information by performing a classification process on the ground truth medical text data, wherein the classification process uses a differentiable model;
obtaining further label information comprising at least one of annotated ground truth label information for the ground truth medical text data and/or synthetic label information for the generated synthetic medical text data; and
performing at least one further data processing task using the predicted ground truth label information and the further label information.

20. A non-transitory memory storing computer-readable instructions that are executable by a processor to:
obtain initial medical data associated with ground truth medical text data, wherein the ground truth medical text data comprises at least partially unstructured medical text data, wherein the initial medical data comprises structured medical text data generated or otherwise obtained from the ground truth medical text data and/or medical image data associated with the ground truth medical text data;
generate synthetic medical text data from the initial medical data using a synthetic text generator, wherein the generated synthetic medical text data comprises at least partially unstructured data;
obtain predicted ground truth label information by performing a classification process on the ground truth medical text data, wherein the classification process uses a differentiable model;
obtain further label information comprising at least one of annotated ground truth label information for the ground truth medical text data and/or synthetic label information for the generated synthetic medical text data; and
perform at least one further data processing task using the predicted ground truth label information and the further label information.
